(19) United States
(12) Patent Application Publication
(10) Pub. No.: US 2005/0049577 A1
(43) Pub. Date: Mar. 3, 2005

(54) Medical device

(76) Inventors: Robert Snell, Suffolk (GB); Sam Martin, Suffolk (GB); Martin Cable, Cambridgeshire (GB); Eleanore King, Northants (GB)

Correspondence Address: JACOBSON HOLMAN PLLC, 400 SEVENTH STREET N.W., SUITE 600, WASHINGTON, DC, 20004, US

(21) Appl. No.: 10/870,436

(22) Filed: Jun. 18, 2004

Related U.S. Application Data
(60) Provisional application No. 60/480,978, filed on Jun. 24, 2003.
(60) Provisional application No. 60/482,140, filed on Jun. 24, 2003.

(30) Foreign Application Priority Data
Jun. 20, 2003 (GB) 0314503.4
Jun. 20, 2003 (GB) 0314504.2

(51) Int. Cl. A61M 27/00; B32B 15/04

(52) U.S. Cl. 604/544; 264/171.16

(57) ABSTRACT

The invention provides a medical device such as a catheter for draining fluids from the body of a living being. The device has a braided portion with crossed filaments which form a braiding angle with a centre axis of the device. The braided portion can be expanded in a direction transverse to the centre axis e.g. in order to anchor the device in the body, e.g. to retain a urinary catheter in the bladder of a patient. The filaments form at least two different braiding angles which thereby facilitate an increased expansion or expansion into a specific shape of the expanded portion and thereby facilitates an improved anchoring of the device in the body.

The present invention relates to a medical device such as a catheter, and to a method of making such a device. The invention further relates to a tube, e.g. to be incorporated into a medical device or to be used in non-medical applications. The device comprises a braided portion which can be reshaped e.g. to anchor the device in the body of a living being.

### BACKGROUND OF THE INVENTION

Medical devices such as catheters with anchoring means for retention in the body of a living being are known. One such device is in general mentioned as a “Foley type catheter” for urinary draining of a bladder e.g. via urethra or via an artificial urinary canal. Foley catheters are provided with an inflatable balloon in an insertable proximal end portion. During use, the catheter is inserted into the urethra, and by inflation of the balloon, the proximal catheter end is anchored against the walls of the urinary canal or in the bladder of the living being. Analogously, tracheal tubes with inflatable balloons for fixation in the trachea during ventilation of a patient, exit. U.S. Pat. No. 4,154,242 discloses another type of a self-retaining catheter comprising an array of wings. To overcome problems e.g. related to the strength of the more traditional catheters, a balloon of the traditional kind have been embedded in a sheath of woven braid, c.f. e.g. U.S. Pat. No. 4,819,751. Whereas the more traditional Foley type catheters depend on a completely sealed pressurized balloon, more advanced anchoring systems are controllable to increase or decrease in radial size without inflation. An example of such a catheter is disclosed in U.S. Pat. No. 4,572,186 wherein a braided cylinder has an adjustable axial length and a structure facilitating an increased radial size upon reduction of its adjustable axial length.

A more advanced catheter is disclosed in U.S. Pat. No. 5,041,093 wherein an elongated flexible tubular member with an inner lumen is provided with a woven tube disposed near the distal end. The woven tube of this catheter is foramenous and axially and radially elastically extensible. To anchor the catheter in the body of a living being, the woven tube is configurable into an over-centre configuration wherein the woven tube forms a cup-like shape.

Irrespective of its superiority over traditional Foley catheters e.g. with respect to strength and durability, the braided or woven catheters show disadvantages e.g. with respect to the extent of expandability and thus the extent of fixation in the body. Due to the mechanical properties of the existing catheters, the desire of expandability of the catheter is normally in contrast to the desire of obtaining sufficient stiffness to avoid folding or kinking of the catheter during handing.

### DESCRIPTION OF THE INVENTION

It is an object of the invention to provide an improvement over the known medical devices. Accordingly, the invention, in a first aspect, provides a medical device having a proximal end for insertion into a body of a living being and an opposite distal end, the device comprising an elongated tube forming a conduit extending in an axial direction, the tube comprising a braided portion with crossed filaments which form a braiding angle with the axial direction, the braided portion being operable between a first configuration and a second configuration wherein the braided portion is expanded in a direction transverse to the axial direction, and in particular to provide specific shapes of the braided portion in the second configuration or to allow a large degree of expansion of the braided portion, the filaments may form at least two different angles with the axial direction for two different locations in the axial direction of the tube. The change in the angle may exist both in the first and in the second configuration.

In one embodiment, the angle changes between two values at two locations along the length of the device, and in another embodiment the angle changes continuously in the axial direction of the device.

In order to further increase the degree of expansion or to form a specific shape of the braided portion in the second configuration, a first part of the braided portion may be located inside a second part of the braided portion when the device is in the second configuration. The first and second parts of the braided portion does not have to be structurally separated, but could form one uniform braided portion, and merely the fold arising by the inverting or rolling of one part of the braided portion into another part of the braided portion defines the transition between the first and second parts of the braided portion. In the first configuration, the braided portion could extend un-folded in the axial direction.

The wording “braided portion” includes in general a tubular portion provided with through-going windows, i.e. openings formed from an outer peripheral surface of the device to the conduit, and e.g. being symmetrically arranged to form a uniform grid of windows. More specifically, the braided portion may comprise cross-braided filaments, i.e. threads which are braided over and under each other. Preferably, the braiding enables the filaments to slide relative to each other. Alternatively, the filaments are arranged in two separate and parallel layers wherein the filaments of one of the layers extend in a direction different from the direction of the filaments of the other layer. In each intersection between a filament of one of the layers and a filament of another layer, the filaments of the two layers may be joined by adhesion. The braided portion could also be constituted by a tubular section with openings forming a mesh-pattern. Irrespective of the type of braiding, the braiding angle, α, which the filaments form with the axial direction, is important for determining the degree of radial expansion and the more precise shape of the braided portion which arises when the braided portion enters the second configuration, and more specifically, when the first part is displaced into the second part of the braided portion. Since the dimension transverse to the axial direction is in inverse sinusoidal relationship to the braiding angle, the change in the angle along the axial direction may be utilised to form various degrees of expansion and expansion into different shapes can furthermore be obtained, thereby possibly facilitating a better fixation of the device in the body.

In the first configuration, i.e. in an extended state wherein the first and second parts of the braided portion is located in axial extension to each other, the braided portion has a generally cylindrical or ovoid shape facilitating insertion into the body. Depending on how the braiding angle is varied, the shape of the resulting funnel can be controlled, and in the second configuration, the braided portion may be provided to form an ovoid shape of a larger dimension transverse to the axial direction, a funnel shape, a tulip-like shape, a disc-shape, a hemispherical shell, a conical shell, a elliptic parabolic shell or any other cup like shell or any other shapes which support retaining of the device in the body. In particular, axially disposed rim portions of the braided portion may form a larger braiding angle than an intermediate centre portion.

The medical device, especially the retention section, may be designed with “shape-memory” such that it will automatically move towards a predetermined shape i.e. towards a relaxed state. In a first embodiment the medical device is designed such that the predetermined shape is the first configuration, i.e. the medical device will have a tendency to move towards the first configuration, but may be moved into the second configuration by axial displacement of the first part of the braided portion into a second part of the braided portion. In some embodiments the first configuration is a configuration wherein the largest dimension of a cross-section of the braided portion is equal or less than the largest dimension of a cross-section of the remaining part of the tube.

In a second embodiment the medical device is designed such that the predetermined shape is the second configuration, i.e. the medical device will have a tendency to move towards the second configuration, but may be moved into the first configuration by axial displacement of the first part of the braided portion out of the second part of the braided portion. In some embodiments the second configuration is a configuration wherein the largest dimension of a cross-section of the braided portion is larger than the largest dimension of a cross-section of the remaining part of the tube.

In a third embodiment the medical device is designed such that the predetermined shape when the braided portion is located inside the remaining part of the tube, e.g. the drainage section, coaxially therewith. When the medical device is located in the body, the second part is displaced out of the remaining part of the medical device to form a medical device in the second configuration, i.e. retained in the body. To operate the medical device between the different configurations, a deployment member could be fastened in the proximal end, e.g. to a proximal tip of the medical device, and extend to the distal end to facilitate manipulation of the proximal end from outside the body. The Deployment rod could e.g. extend inside the lumen. The tip could be shaped as a Nelaton tip or as a Tiemann tip, or the tip could have the form of an open ring, e.g. with a smoothly rounded part extending in a forward direction to form a proximal end of the device and thus to facilitate comfortable insertion of the device into the body. The open ring may facilitate entrance of larger bodies, e.g. coagulated blood etc. into the conduit, and for that reason, an area of the opening in the centre of the ring could preferably constitute 50% or more of a cross sectional area of the conduit.

The mesh openings or “windows” may form paths between the outer surface of the braided portion and the conduit e.g. to drain body fluids such as urine into the conduit. One or more additional opening could be located either in a distal part of the second part of the braided portion or in an optional proximal tip-part of the device. To drain fluids from the body, the device could be designed as a medical catheter, e.g. a urinary catheter or in general as a catheter for draining body fluids from a body lumen. As a urinary catheter, the device could be adapted for insertion through the urethra into the bladder of the living being, or as a suprapubic catheter in which case the device is passed surgically into the bladder through a surgical incision in the abdominal muscle wall. Until now, it has been described that the medical device comprises an elongated tube with a braided portion with crossed filaments which form a braiding angle with the axial direction, and that the braided portion is operable between two configurations. In fact, the entire medical device could be made from a braided material, but in this case, a part of the braiding may serve as a retention section which part is therefore operable between the two configurations. The remaining part of the entirely braided device could serve as e.g. as a drainage section for draining body fluids or in general serve to establish a passage into the body. In the drainage section, the braiding could serve to reinforce a composite of the braiding filaments and a matrix material of the kind described later. In the table below, it is indicated how the use of a braided drainage section can increase the cross section of the conduit in a catheter.

| | Traditional | Catheter with |
|---|---|---|
| | silicone | a braided |
| | catheter: cross | drainage section: cross |
| Ch | sectional area | sectional area |
| size | [mm²] of conduit | [mm²] of conduit |
| 8 | 1.3 | 2.3 |
| 10 | 3.1 | 4.2 |
| 12 | 3.8 | 7.1 |
| 14 | 4.9 | 10.8 |
| 16 | 3.8 | 14.5 |
| 18 | 8.0 | 19.6 |

To facilitate comfortable insertion or to reduce adherence of body tissue to the surface of the device, at least a part of the device, e.g. the braided portion or the second part of the braided portion or the tip-part may have an outer surface, i.e. a surface towards the body tissue when the device is inserted into the body, which surface has a low surface friction characteristic compared to other parts of the device. To provide the low friction characteristic, the medical device, e.g. the braided portion thereof may have a hydrophilic surface, e.g. provided by a hydrophilic coating of the surface, e.g. a coating comprising polyvinylpyrolidone. In one embodiment, namely when the first part of the braided portion is located inside the second part of the braided portion, the low surface friction may in particular be provided on the second part of the braided portion. During retention inside the body, the body tissue is thereby protected by the low friction character of the second part. A hydrophilic coating may further reduce irritation of the body tissue, e.g. mucosa. If a hydrophilic coating is applied to the braided portion, the coating may incorporate an anti-infective compound or a compound which counteracts ingrowth.

The filaments could e.g. be made from polyester, polyamide, polyalkane, polyurethane, PET, PBT, Nylon, PEEK, PE, Glass Fibre, Metal Wire or Acrylic materials or any composition of the mentioned materials. A preferred material would be PET or polyester.

The device may include a matrix material, e.g. any medical grade polymer that can be dissolved in a solvent or be manufactured as a polymer emulsion. Examples of theses are polyurethane, polyurethane dispersions, acrylic, PVC, block copolymers (SIS SBS) etc, natural rubber, silicone, neoprene, nitrile or compositions thereof. Polyurethane, acrylic, PVC, block copolymers (SIS SBS) etc, natural rubber, silicone, or EPO or compositions thereof, could be used if the device is made by extrusion or injection moulding.

In a second aspect, the invention provides a tube forming a conduit extending in an axial direction, which tube comprises a braided portion with crossed filaments which form a braiding angle with the axial direction, the braided portion being operable between a first configuration and a second configuration forming different sizes of the tube in a direction transverse to the axial direction, and in particular, a tube wherein the braiding angle changes along the length of the tube, e.g. between two different braiding angles or continuously along the length of the tube.

In a third aspect, the invention provides a method of forming a tube forming a conduit extending in an axial direction, which tube comprises a braided portion with crossed filaments which form a braiding angle with the axial direction, the braided portion being operable between a first configuration and a second configuration forming different sizes of the tube in a direction transverse to the axial direction, said method comprising the step of braiding the filaments together at a braiding angle that changes along the length of the tube, e.g. by operating a braiding machine at varying axial braiding speed or at varying speed of a braiding table. to vary the angle either the axial speed needs to remain constant and the table speed varied or the table speed remains contant and the axial speed varies.

In a fourth aspect, the invention provides the use of a medical device according to the first aspect for improving fixation of the device in the body of a living being. Since the braiding angle is varied along the length of the catheter, an improved fixation can be obtained, e.g. in the body of a human being.

### DETAILED DESCRIPTION OF THE INVENTION

In the following, a preferred embodiment of the invention will be described in further details with reference to the drawing in which:

FIG. 1 illustrates a urinary catheter according to the invention, and in the first configuration,

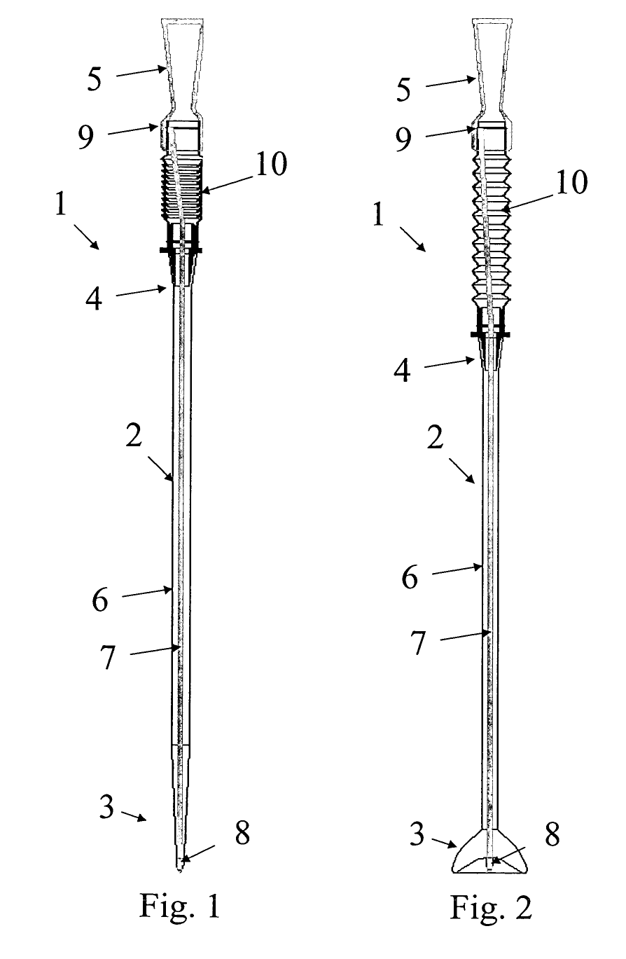

FIG. 2 illustrates the catheter of FIG. 1 in the second configuration,

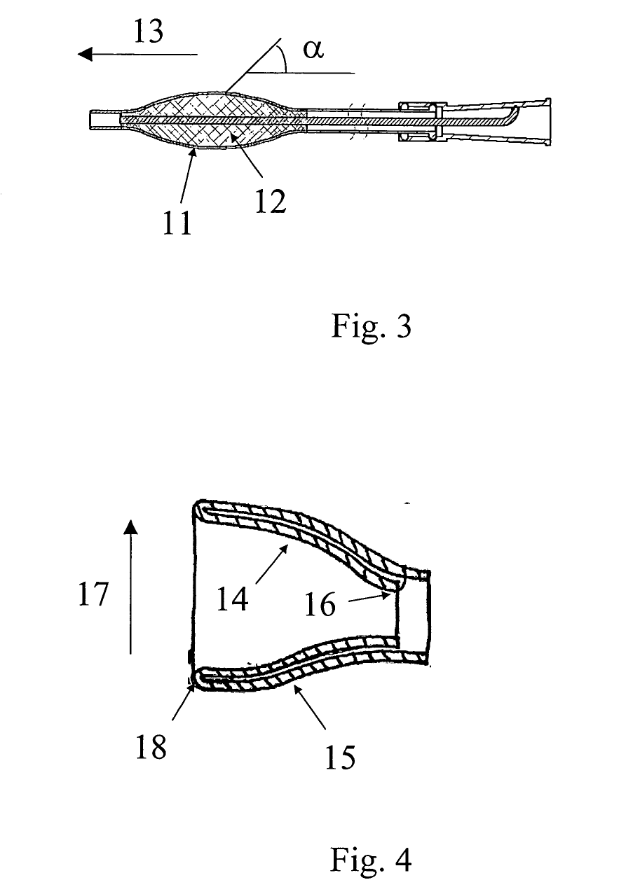

FIG. 3 shows an enlarged view of the braided portion in the first configuration,

FIG. 4 shows an enlarged view of the braided portion in the second configuration,

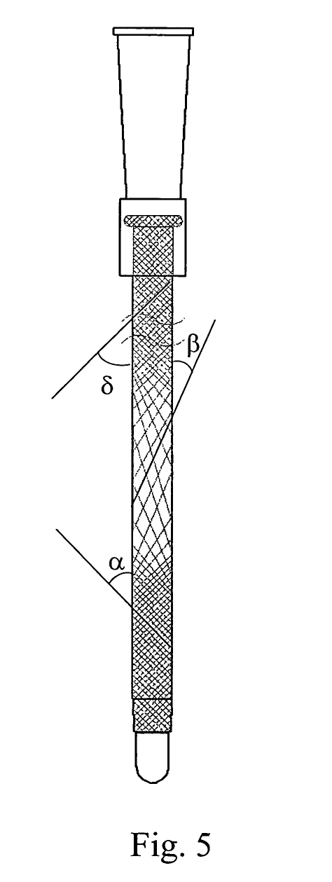

FIG. 5 shows a braiding with tree different braiding angles,

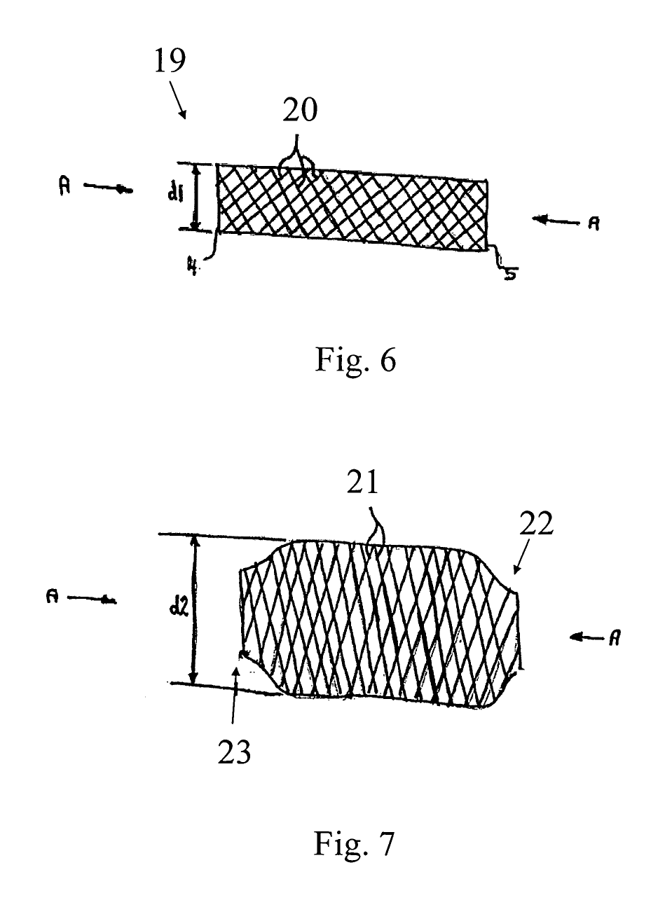

FIGS. 6 and 7 show side view of a tube formed from filaments,

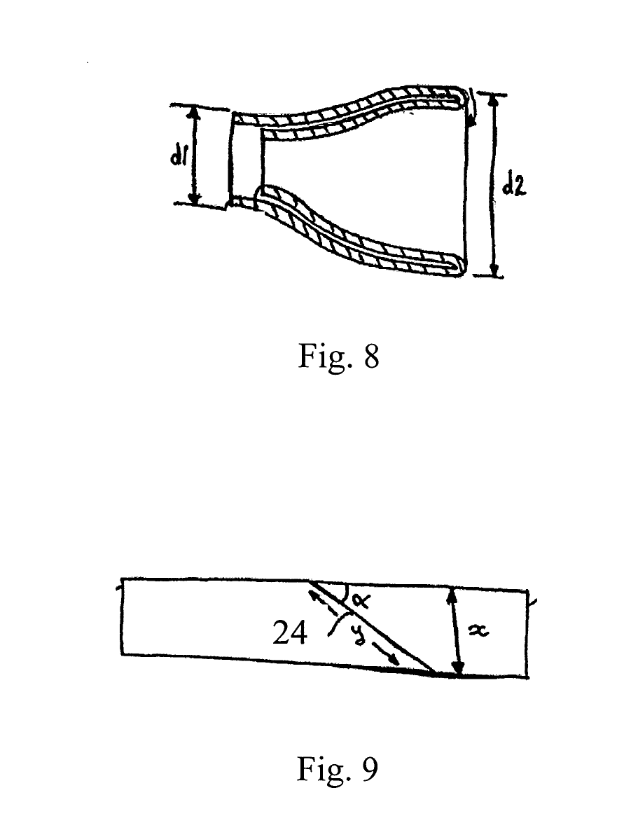

FIG. 8 shows a view of the tube in FIGS. 6 and 7 in a configuration wherein a first part is located inside a second part of the braiding,

FIG. 9 shows the braiding angle of one single filament,

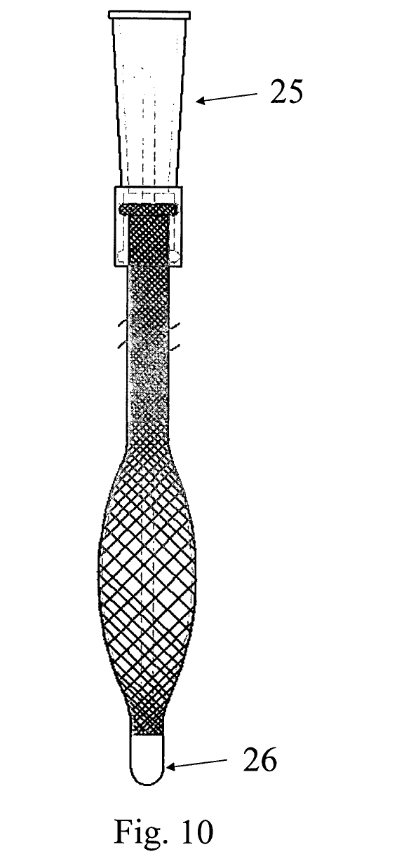

FIG. 10 shows a view of a catheter made with a braiding extending from a distal connector to the proximal end,

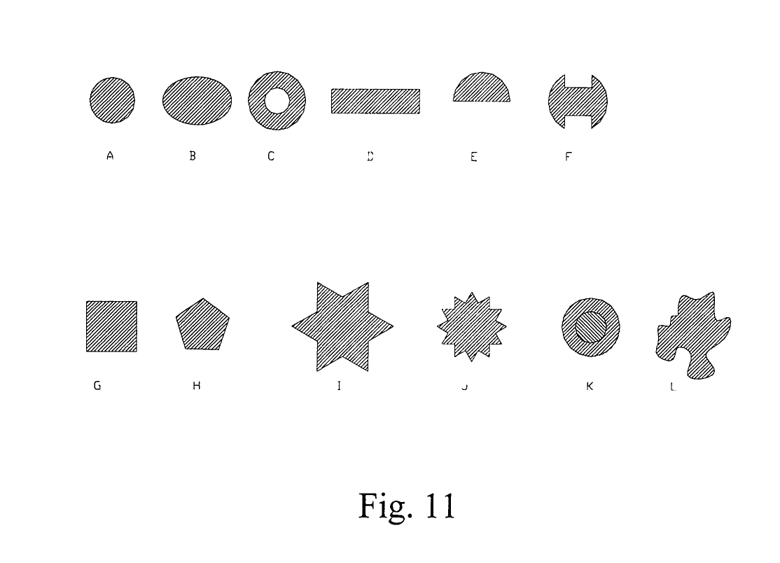

FIG. 11 shows various cross sections of filaments, and

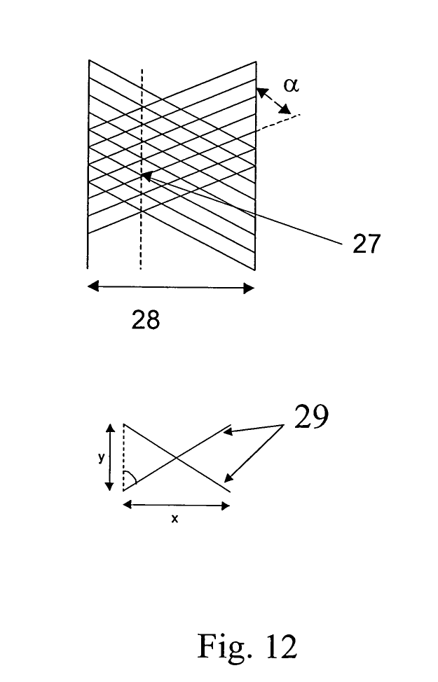

FIG. 12 shows details of single filaments of a braiding.

The device could be used for transporting fluids or substances into or out of a body, e.g. for gastro content aspiration. The device could be applied subcutaneously or through insertion of the catheter into a natural or artificial opening in the body, or the medical device could be applied for stent delivery, e.g. for placing a stent within the prostatic urethra, or in general for draining fluids from a natural or artificial body lumen, for anal insertion or for insertion into the gastrointestinal region, e.g. with the purpose of fixating a camera or surgical instruments inside the body or in general to establish a passage into the body. In the remaining part of the description, the medical device is described with reference to a catheter, and in particular to a catheter for urinary drainage, i.e. wherein the catheter is inserted into a natural or artificial urinary canal e.g. urethra, and into a bladder for draining urine.

In FIGS. 1 and 2 the catheter is outlined in its full length in a collapsed configuration, cf. FIG. 1 and in an expanded configuration, cf. FIG. 2. As illustrated in FIG. 1, the catheter 1 comprises an elongated flexible tube 2 having a proximal end 3 for insertion into the body of a living being, and comprising a braided portion which facilitate anchoring in the body. In its axially opposite end, the catheter forms a distal end 4 with a connector 5 from which the drained fluid is disposed. Via the connector, fluid can be drained into a connected bag for collecting body fluids, or the tube can be extended by an extension tube. FIG. 1 shows the catheter in its full length in a collapsed, first, configuration for insertion into the body, and FIG. 2 shows the catheter in its full length in an expanded, second, configuration for retention in the body. In the expanded configuration, a first part of the braided portion is drawn into the conduit whereby a funnel shaped termination of the catheter is formed. The catheter forms a conduit 6 extending in an axial direction for draining the body fluid from the proximal end to the distal end. In the conduit, the catheter has a deployment member 7 fastened to the proximal tip 8 and to a point 9 in the vicinity of the connector 5. To manipulate the catheter between the first and the second configurations, a corrugated part, e.g. a bellow shaped member 10 is inserted between the two points in which the rod is fastened to the catheter. The bellow shaped member 10 allows axial displacement of the deployment member, and thus of the proximal tip 8 relative to the remaining part of the catheter.

FIG. 3 shows an enlarged view of the braided portion 11 in which the crossed filaments 12 mutually form a braiding angle indicated by α.

In FIG. 3, the catheter is in a configuration for insertion into the body, i.e. wherein the entire braided portion extends in one and the same direction indicated by the arrow 13.

FIG. 4 shows the catheter in a situation wherein a first part 14 of the braided portion is located inside a second part 15 of the braided portion, meaning that the end 16 of the tube has been inverted or rolled inward of itself so that it has become ensheathed by the remainder of the tube. The exact same configuration is achieved by inverting or rolling the end 16 outward over itself so that the tube becomes partially ensheathed. As shown, the catheter expands in a direction transverse to the axial direction (visualised by the arrow 17), thereby facilitating retention of the catheter inside the body. In FIG. 4, the catheter is provided with a braiding which forms a funnel shaped proximal end of the catheter. The funnel has a mouth portion 18 which is the point at which the diameter of the funnel is at its maximum value. This diameter is the same as the maximum diameter of the tube when the tube is radially expanded and is equal to d2, cf. also FIG. 8. FIGS. 4 and 8 do not disclose an specific smooth tip. The tip could, however be formed as a Nelaton or a Tiemann tip or as an open ring or in fact as a regular catheter tip with openings for draining fluid into the conduit.

In FIG. 5, it is shown how the filaments of the braiding change between three different angles, one angle, α, in the proximal end of the braided portion, a second angle, β, in an intermediate part of the braided portion and a third angle δ in the opposite distal end of the braided portion. In the disclosed embodiment, α equals δ.

FIGS. 6 and 7 show side views of a tube 19 formed from filaments 20 which are braided together so that they can all move relative to each other and allow the tube to expand radially from a diameter d1 in a relaxed state to a maximum diameter d2 when an axial compressive force is applied in the direction of arrow “A”. FIG. 7 shows the tube of FIG. 6 in which a portion 21 of the tube has reached its maximum diameter d2 following compression. It will be appreciated that when the compressive force is removed or released, the tube returns to its original relaxed state in which the diameter of the tube is equal to d1. The tube has axially opposite ends 22, 23. FIG. 8 shows the tube in the situation wherein a first part of the braiding is located inside a second part of the braiding, and wherein d1 and d2 indicate the radial dimension in the distal and proximal end of the braided portion, respectively.

The braiding angle is the angle of the braided filaments relative to the longitudinal axis of the tube. The braiding angle is shown in the unexpanded tube of FIG. 9 (only one braid filament 24 is shown for the purposes of clarity). The braiding angle is represented by “α”, the angled length of the braid across the diameter of the tube is designated by “y” and the diameter of the tube before any radial expansion is designated by “x”.

If it is assumed that length “y” is the theoretical maximum diameter that the tube can have when expanded, and that the initial diameter “x” of the tube in its relaxed state is equal to 1, then it is possible to calculate the braiding angle that is required in order to provide a funnel having a required maximum diameter using Pythagoras's Theorem which states that:
Sin α=x/y

Assuming that the diameter of the tube in its relaxed state is equal to 1, then:

Sin α=1/y, and so the maximum diameter of the funnel is given by:
y=1/Sin α.

It is therefore apparent that the braiding angle and the maximum diameter of the tube or funnel have an inverse sinusoidal relationship.

At a braiding angle of 15 degrees, the diameter of the expanded tube or the maximum diameter of the funnel is 3.864 times the diameter of the tube before expansion. Similarly, at a braiding angle of 30 degrees, the diameter of the expanded tube or the maximum diameter of the funnel is twice the diameter of the tube before expansion. When the braiding angle is 60 degrees, the maximum diameter of the tube when expanded or the maximum diameter of the funnel is 1.154 times the diameter before expansion.

By relying on the relationship between the braiding angle and the maximum diameter of the funnel, it is possible to select a braiding angle to provide a tube having a known maximum diameter and, consequently, to provide a funnel having a large diameter.

It will also be appreciated that by making a tube with a non-uniform braiding angle along its length, the shape of the funnel can be altered so that shapes other than just conically shaped funnels can be formed.

FIG. 10 shows a view of a catheter with a large braided portion extending from the connector 25 to the proximal end 26, and wherein a part of the braiding forms the braided portion according to the present invention. The proximal end is provided with a smoothly rounded tip which facilitate comfortable insertion of the catheter, e.g. in a urinary tract.

FIG. 11 shows various cross sectional shapes of the filaments used for the braiding. 11A shows a round cross section, commonly supplied e.g. by Rhodia or Swicofil, c.f. www.rhodia.com, 11B is an oval cross section which allows for large cross section of material with a reduced cross over height, and which may facilitate a reduced wall thickness. 11C is a tubular filament which can be applied to many different shapes, and which provides similar structural strength but with less material in the filament. 11D is a flat tape filament with characteristics similar the filament of FIG. 11B. 11E is a hemispherical cross section, with characteristics similar the filament of FIG. 11B. 11F is a cut out round which can reduce cross over height and which can increase the surface area for better incorporation into the polymer matrix. 11G is a square cross section, and 11H is a polygon of any number of sides. 11H can be applied to provide better filament nesting. 11I & 11J show star shapes which could be applied to increase the surface area for better incorporation into the polymer matrix. 11K shows two different materials in one filament, outer material to provide good matrix adhesion, inner material to provide stiffness (can be applied to all shapes). Finally, 11L shows a random shape which increases the surface area for better incorporation into the polymer matrix.

With reference to FIG. 12, wherein numeral 27 indicates a braid crossover, 28 indicates the diameter of a braided tube and 29 indicates filaments, the following text describes how to calculate the braiding.

Calculation of Picks Per Inch

The angle of the braid is dependent on the number of yarns/filaments (N), the diameter of the rod (D) the speed of the winding table (s) and the speed of the transition of the rod through the braiding head. The speed of winding table, the speed of movement of the former rod and the number of yams determine the number of picks per inch.

By considering a unit cell of length x and height y, it can be seen that the braid angle (α) is given by tan

$$α = \frac{x}{y} .$$

The unit cell width, x, can be expressed in terms of the machine and mandrel as follows:

$$x = \frac{2 π R}{N}$$

where R=radius of the mandrel and N=number of carriers on the braiding machine.

From the diagrams it can be seen that the number of picks per inch is inversely proportional the unit cell height, y when given in inches.

$$Number of picks per inch = \frac{1}{y} = \frac{tan a}{x}$$

Therefore, picks per inch can be expressed as:

$$ppi = \frac{N tan a}{2 π D}$$

Where D=diameter of forming mandrel, in inches.

The above equation has been modified to include the diameter of the monofilament. For very fine monofilaments, the contribution of the diameter is negligible but for larger monofilaments it must be taken into account as follows:

$$ppi = \frac{N tan a}{2 π ( D + 2 d )}$$

Where d=monofilament diameter, in inches.

Filament Spacing

In order to consider the theoretical maximum spacing between filaments there are several factors to consider.

For a given ppi (as calculated above and measured from the centre of the filaments) the diameter of the filament (d) will determine the spacing between the extreme quadrants of the filaments.

Braiding Angle α

Filament Diameter d

Perpendicular spacing between filaments P

The angular thickness of the filament

$$( b ) = \frac{d}{cos a}$$

Each angular gap between filaments for an inch

$$( g ) = \frac{ppi \times b}{ppi}$$

Perpendicular spacing between filaments (P)=g×cos α

This calculation is true for filaments with a round cross section, cross sectional geometry will have some effect on the calculated ppi value.

In the following, one example of a manufacturing process is described with reference to manufacturing of a catheter.

A forming rod which has the external diameter required for the manufacture of a catheter with the equivalent internal diameter is taken. The forming rod is partially dipped in a solution of polyurethane, where the speed of dipping withdrawal, the number of dipped layers and the total solids of the polyurethane solution dictate the film thickness deposited on the forming rod. The forming rod is coated to provide a dry film thickness of between 20 and 80 microns single wall thickness. Once the film formed on the forming rod is sufficiently dry the rod is passed through a braiding machine where, filaments are braided onto the forming rod at a given angle. The angle of the braiding is determined by the size of the forming rod, the speed of the braiding table, the axial speed of the part through the table, the number of filaments on the braiding machine and the filament size. The catheter is formed of at least 2 regions with different braid angles. The drainage section is formed from an elongated braid section with an angle in the region of 54.3 degrees to provide maximum flexibility and kink resistance. The second section is braided at a significantly lower angle to allow for the expansion of the retention means. The braided forming rod is partially dipped in a solution of polyurethane, where the speed of dipping withdrawal, the number of dipped layers and the total solids of the polyurethane solution dictate the film thickness deposited on the forming rod. The thickness of these final dips will determine the outer diameter of the catheter drainage tube. Once the polyurethane film has been dried the braided tube is removed from the forming rod by axially compressing the braided tube and sliding the forming rod from within the tube. This can be further facilitated by the use of release coatings on the forming rod or additives within the polymer dipping solution. Alternatively the forming rod can be extensible, where the forming rod decreases in diameter during axial extension, facilitating the removal of the reduced diameter rod from the internal lumen of the tube.

The connector and deployment means is then joined to the tube section, with the deployment means traversing through the internal lumen of the drainage section. The deployment means is then attached to the proximal end of the retention section in its extended configuration by thermal welding.

A particular application of the invention is in the manufacture of catheters for medical use. For example, a catheter may be provided with a retention section for retaining the catheter in position in the body of the patient once it has been inserted. The retention section may be formed from the braided tube of the invention which is maintained in the form in which it is illustrated in FIG. 3 during insertion and removal of the catheter and, may be manipulated so that it assumes the funnel shaped configuration shown in FIG. 4, once inserted. The funnel shape assists in retaining the catheter in position in, for example, the urinary bladder of the patient and also directs bodily fluids such as urine, in the case of a urinary catheter, into the catheter itself. The present invention realises that the shape of the funnel can be altered by changing the braiding angle of the retention section. The surgeon or other responsible member of the medical staff can then pick an appropriate catheter to suit the patient or bodily cavity in which the funnel is to be located. In addition, a tube with a braided portion according to the invention can be applied in numerous non-medical applications.

Many modifications and variations of the invention falling within the terms of the appended claims will be apparent to those skilled in the art and the foregoing description should be regarded as a description of a preferred embodiment only.

1: A medical device having a proximal end for insertion into a body of a living being and an opposite distal end, the device comprising an elongated tube forming a conduit extending in an axial direction, the tube comprising a braided portion with crossed filaments which form a braiding angle with the axial direction, the braided portion being operable between a first configuration wherein it has a first size in a direction transverse to the axial direction and a second configuration wherein it has a second size in the direction transverse to the axial direction, the second size being larger than the first size.

2: A medical device according to claim 1, wherein the filaments form at least two different angles with the axial direction for two different locations in the axial direction of the tube.

3: A medical device according to claim 1, wherein a first part of the braided portion is located inside a second part of the braided portion when the device is in the second configuration.

4: A medical device according to claim 3, wherein the proximal end of the device forms a largest size transverse to the axial direction when the device is in the second configuration, which size is in inverse sinusoidal relationship with the braiding angle.

5: A medical device according to claim 1, wherein the braided portion has axially disposed rim portions and an intermediate portion between the rim portions, and wherein the braiding angle increases from the intermediate portion towards at least one of the rim portions.

6: A medical device according to claim 1, wherein the conduit is dimensioned for transfer of fluids.

7: A medical device according to claim 6, forming a catheter.

8: A medical device according to claim 1, wherein at least a part of the braided portion comprises a matrix material.

9: A medical device according to claim 1, wherein at least a part of the braided portion comprises a hydrophilic surface.

10: A tube forming a conduit extending in an axial direction, which tube comprises a braided portion with crossed filaments which form a braiding angle with the axial direction, the braided portion being operable between a first configuration wherein it has a first size in a direction transverse to the axial direction and a second configuration wherein it has a second size in the direction transverse to the axial direction, the second size being larger than the first size.

11: A tube according to claim 10, wherein the filaments form at least two different angles with the axial direction for two different locations in the axial direction of the tube.

12: A tube according to claim 11, wherein a first part of the braided portion is located inside a second part of the braided portion when the tube is in the second configuration.

13: A method of forming a tube forming a conduit extending in an axial direction, which tube comprises a braided portion with crossed filaments which form a braiding angle with the axial direction, the braided portion being operable between a first configuration wherein it has a first size in a direction transverse to the axial direction and a second configuration wherein it has a second size in the direction transverse to the axial direction, the second size being larger than the first size, said method comprising the step of braiding the filaments together at a braiding angle that changes along the length of the tube.

14: A method according to claim 13, wherein the change in braiding angle is achieved by operating a braiding machine at varying axial braiding speed or by varying the rotational speed of a braiding table.

15: The use of a medical device according to claim 1, for improving fixation of the device in the body of a living being.
